(19) United States
(12) Patent Application Publication
(10) Pub. No.: US 2018/0313801 A1
(43) Pub. Date: Nov. 1, 2018

(54) METHOD FOR DIAGNOSING RHEUMATOID ARTHRITIS

(71) Applicant: Board of Supervisors of Louisiana State University and Agricultural and Mechanical College, Baton Rouge, LA (US)

(72) Inventors: Christopher KEVIL, Shreveport, LA (US); Richa DHAWAN, Shreveport, LA (US); Alexis GUICE, Shreveport, LA (US)

(21) Appl. No.: 15/769,976

(22) PCT Filed: Oct. 21, 2016
(86) PCT No.: PCT/US2016/058126

§ 371 Date: Apr. 20, 2018

Related U.S. Application Data
(60) Provisional application No. 62/244,354, filed on Oct. 21, 2015.

(51) Int. Cl. G01N 33/00; G01N 31/22; G01N 33/49

(52) U.S. Cl. CPC G01N 33/0044; A61K 31/655; G01N 33/4925; G01N 31/223

(57) ABSTRACT

This disclosure describes the use of plasma hydrogen sulfide metabolites as biomarkers for Rheumatoid arthritis, as well as the potential use of these metabolites as therapeutic efficacy targets.

The present invention claims priority to U.S. Provisional Patent Application No. 62/244,354 filed Oct. 21, 2015, which is incorporated by reference into the present disclosure as if fully restated herein. U.S. Patent application Ser. No. 14/780,799, filed Mar. 28, 2014, and U.S. Pat. No. 9,465,024, filed Mar. 3, 2014 are also incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### FIELD OF THE INVENTION

The present invention relates to the diagnosis of Rheumatoid arthritis (RA) through detection of an elevated level of hydrogen sulfide (H₂S) in plasma, including bound sulfane, sulfur, and total sulfide levels, and to the use of plasma hydrogen sulfide metabolite bioavailability as a biomarker of RA or for therapeutic efficacy of treatment for RA.

### BACKGROUND OF THE INVENTION

RA is a chronic systemic autoimmune inflammatory disease that affects between 0.5 and 1% of adults in the developed world, with between 5 and 50 per 100,000 people newly developing the condition each year. The primary target organ in RA is the synovial membrane. Changes include increased vascularity and infiltration with immune inflammatory cells. While RA predominantly affects the joints, it may also lead to systemic manifestations. Most of the research in RA has been focused on pathogenesis which has allowed for the development of new therapies to target the proinflammatory cytokines that play a key role in synovitis and tissue destruction.

Related references in the field teaches in patients with (RA) the presence of increased hydrogen sulfide (in its broadest sense) bioavailability in synovial fluid from joints but normal levels of hydrogen sulfide in plasma. See Whiteman, M., Haigh, R., Tarr, J. M., Gooding, K. M., Shore, A. C. and Winyard, P. G. (2010), “Detection of hydrogen sulfide in plasma and knee-joint synovial fluid from rheumatoid arthritis patients: relation to clinical and laboratory measures of inflammation,” Annals of the New York Academy of Sciences, 1203:146-150. Likewise, other recent studies indicated that hydrogen sulfide (in its broadest sense) bioavailability was increased in synovial fluid of joints of RA patients, but that hydrogen sulfide was not increased in the plasma of RA patients. See, e.g Muniraj, N., Stamp, L. K., Badiei, A., Hegde, A., Cameron, V. and Bhatia, M. (2014), “Hydrogen sulfide acts as a pro-inflammatory mediator in rheumatic disease,” International Journal of Rheumatic Diseases, doi: 10.1111/1756-185X.12472. However, accessing the synovial fluid of patients to determine RA status is highly discomforting and painful for the patients, dramatically limiting the efficacy of such tests. Thus, there is an ongoing and unmet need for improved methods for diagnosing RA, and in particular for use in diagnosis before the diseases progresses to a point where current therapeutic approaches are inadequate.

### SUMMARY OF THE INVENTION

Wherefore, it is an object of the present invention to overcome the above mentioned shortcomings and drawbacks associated with the prior art.

New analytical methods for measurement of various, inter alia, biochemical H₂S forms have recently been developed by inventors having a common assignee as the present disclosure. See U.S. patent application Ser. No. 14/780,799 and U.S. Pat. No. 9,465,024. No information is available in the art on the amounts of these various biochemical forms of H₂S in patients with autoimmune diseases. To the inventors' great surprise, and contrary to the teachings and current understanding in the field, the inventors' discovered that patients with RA have significant increased levels of bound sulfane sulfur and total sulfide levels in their plasma. These findings were possible using the new sensitive analytical HPLC methodologies described in U.S. patent application Ser. No. 14/780,799 and U.S. Pat. No. 9,465,024.

Another object of the present invention is to diagnose RA in a human patient.

A further object of the present invention is to diagnose RA at a pre-symptomatic early stage of the disease and/or to diagnose a pre-RA condition.

Yet another object of the present invention is to monitor an efficacy of treatment for RA disease.

A still further object of the present invention is to provide a diagnostic test or as a test to determine therapeutic efficacy for RA.

A further another object of the present invention is to diagnose RA in a patient by testing a patient's plasma sample with an analytical instrument or Hydrogen Sulfide Detecting Apparatus, as described in U.S. patent application Ser. No. 14/780,799 and/or determining the levels of one or more of free sulfide, acid labile sulfide, bound sulfane, and total sulfide in a patent's plasma, including by methods described in U.S. Pat. No. 9,465,024.

A first embodiment of the invention relates to a method for diagnosing

Rheumatoid arthritis in a human patient comprising obtaining a plasma sample from the patient, using an analytical instrument to determine a level of H2S in the sample; and diagnosing a patient with Rheumatoid arthritis when the level of H2S in the sample is above a control level of H2S. According to a further embodiment the level of H2S in the sample and level of H2S in the control refers to one of a level of acid labile sulfide, total sulfide, and both acid labile sulfide and total sulfide. According to a further embodiment the method further comprises diagnosing a patient with Rheumatoid arthritis when the level of acid labile sulfide in the plasma is above one of 0.004, 0.005, 0.006, and 0.007 pmol sulfide/mg protein. According to a further embodiment the method further comprises diagnosing a patient with Rheumatoid arthritis when the level of total sulfide in the plasma is above one of 0.014, 0.015, 0.016, 0.017, 0.018, 0.019, and 0.020 pmol sulfide/mg protein. According to a further embodiment the method further comprises diagnosing a patient with Rheumatoid arthritis when the level of acid labile sulfide in the plasma is above one of 0.004, 0.005, 0.006, and 0.007 pmol sulfide/mg protein and the level of total sulfide in the plasma is above one of 0.014, 0.015, 0.016, 0.017, 0.018, 0.019, and 0.020 pmol sulfide/mg protein. According to a further embodiment the method further comprises diagnosing a patient with Rheumatoid arthritis when the level of acid labile sulfide in the plasma is above one of 0.001, 0.002, 0.003, and 0.004 pmol sulfide/mg protein above the level of acid labile sulfide in the control. According to a further embodiment the method further comprises diagnosing a patient with Rheumatoid arthritis when the level of total sulfide in the plasma is above one of 0.001, 0.002, 0.003, 0.004, 0.005, 0.006, and 0.007 pmol sulfide/mg protein above the level of total sulfide in the control. According to a further embodiment the method further comprises diagnosing a patient with Rheumatoid arthritis when the level of acid labile sulfide in the plasma is above one of 0.001, 0.002, 0.003, and 0.004 pmol sulfide/mg protein above the level of acid labile sulfide in the control and the level of total sulfide in the plasma is above one of 0.001, 0.002, 0.003, 0.004, 0.005, 0.006, and 0.007 pmol sulfide/mg protein above the level of total sulfide in the control. According to a further embodiment the method further comprises diagnosing a patient with Rheumatoid arthritis when the level of acid labile sulfide in the plasma one of 10%, 20% 40%, 80%, 100%, 150%, and 175% greater than the level of acid labile sulfide in the control. According to a further embodiment the method further comprises diagnosing a patient with Rheumatoid arthritis when the level of total sulfide in the plasma one of 10%, 20% 30%, 40%, 50%, and 60% greater than the level of total sulfide in the control. According to a further embodiment the method further comprises diagnosing a patient with Rheumatoid arthritis when the level of acid labile sulfide in the plasma one of 10%, 20% 40%, 80%, 100%, 150%, and 175% greater than the level of acid labile sulfide in the control and the level of total sulfide in the plasma one of 10%, 20% 30%, 40%, 50%, and 60% greater than the level of total sulfide in the control. According to a further embodiment the method further comprises the patient is previously suspected to have Rheumatoid arthritis based on diagnostic criteria in addition to the elevated level of H2S, including joint pain.

Another embodiment of the invention relates to a method for diagnosing Rheumatoid arthritis in a human patient comprising obtaining a plasma sample from the patient, using an analytical instrument to determine a level of H2S in the sample, wherein a level of H2S refers to a level of one or more of free sulfide, acid labile sulfide, bound sulfane, and total sulfide, and diagnosing a patient with Rheumatoid arthritis when the level of one or more or all of free sulfide, acid labile sulfide, bound sulfane, and total sulfide in the sample is above a respective control level of free sulfide, acid labile sulfide, bound sulfane, and total sulfide.

Yet another embodiment of the invention relates to a method for diagnosing and treating Rheumatoid arthritis in a patient comprising analyzing a patient plasma sample for an elevated level of H2S, wherein the patient is diagnosed with RA if an elevated level of H2S is detected; and administering pharmacologically effective dose of a therapeutic agent to the diagnosed patient. According to a further embodiment the therapeutic agent is one of a steroid, a corticosteroid, a disease-modifying antirheumatic drug, a biologic, a JAK inhibitor, and a nonsteroidal anti-inflammatory drug.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings, as graphical representations of experimental data, are drawn to scale. The invention will now be described, by way of example, with reference to the accompanying drawings in which:

FIG. 1 is a graph of plasma H₂S levels for individuals with RA and for individuals without RA (as Control) for free H₂S, acid labile H₂S, bound sulfane H₂S, and total sulfide H₂S according to a first example;

FIG. 2A is a graph of acid labile H₂S for individuals with RA and for individuals without RA (as Control) according to a second example; and

FIG. 2B. is a graph of total sulfide H₂S for individuals with RA and for individuals without RA (as Control) according to the second example.

### DETAILED DESCRIPTION OF THE INVENTION

The present invention will be understood by reference to the following detailed description, which should be read in conjunction with the appended drawings. It is to be appreciated that the following detailed description of various embodiments is by way of example only and is not meant to limit, in any way, the scope of the present invention.

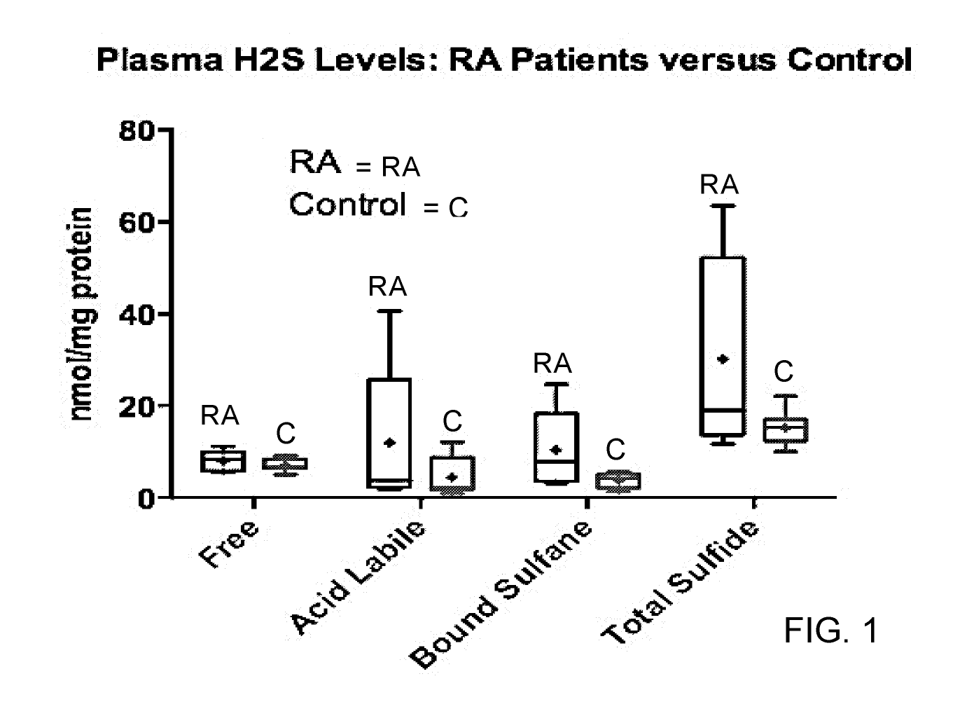

Turning now to FIG. 1, a brief description concerning the various components of the present invention will now be briefly discussed.

Materials & Methods: Patients were stratified into two main cohorts: first, eleven (11) patients without RA, who served as normal controls, and second, five (5) patients with RA were defined by American College of Rheumatology criteria. Arterial blood samples (10 mL) were collected via venipuncture and were prepared for detecting free H₂S by centrifuging. The plasma was transferred to a PCR tube containing Tris-HCL and MBB solution. The solution was incubated in a hypoxic chamber at room temperature. The reaction was stopped by sulfosalicylic acid solution to precipitate the protein which was then vortexed and placed on ice. The tubes were centrifuged and the supernatant was transferred to an HPLC vial.

The supernatant was subsequently injected into the Reversed-phase high-performance liquid chromatography (RP-HLC) system with an Agilent Eclipse XDB-C18 column equilibrated with 15% CH₃CN in water containing 0.1% (v/v) TFA. Monobromobimane and sulfide-dibimane were separated using the gradient of two mobile phases: (A) water containing 0.1% (v/v) TFA and (B) 99.9% CH3CN, 0.1% (v/v) TFA at a flow rate of 0.6 mL/min. The amount of H₂S (from linear plots of the HPLC peak areas of sulfide-dibimane) versus known concentration of sulfide solution was measured. The values obtained were multiplied by 6.6 to account for dilutions prior to analysis.

Samples were prepared for detecting acid-labile sulfide and bound sulfane sulfur in plasma using two sets of BD vacutainer tubes. Phosphate buffer was added to one tube and phosphate buffer with TCEP was added to the other tube. The tubes were incubated on the nutator and the solution is then removed through the cap with a spinal needle. The sulfide gas is trapped by adding 500 microliters of 100 mM Tris-HCl buffer into the BD vacutainer tube, using a 30G needle and the sample is further incubated on the nutator. The solution is removed and the sulfide level in the vacutainer is measured by the MBB method. The remaining blood/plasma sample is frozen in liquid nitrogen and stored for future IRB approved research.

Discussion: As shown in the Figure, results of the RP-HLC analysis demonstrated that overall levels of total plasma H₂S in RA patients were significantly higher than the levels of plasma H₂S in the control population (p=0.02). Furthermore, the levels of bound H₂S were also significantly higher in the RA patient population (p=0.01). While the level of acid labile plasma H₂S appeared to vary greatly between the two populations, from this example, the degree of difference had a p=0.08. Furthermore, from this example, levels of free plasma H₂S among the RA patients and control populations had a p=0.18.

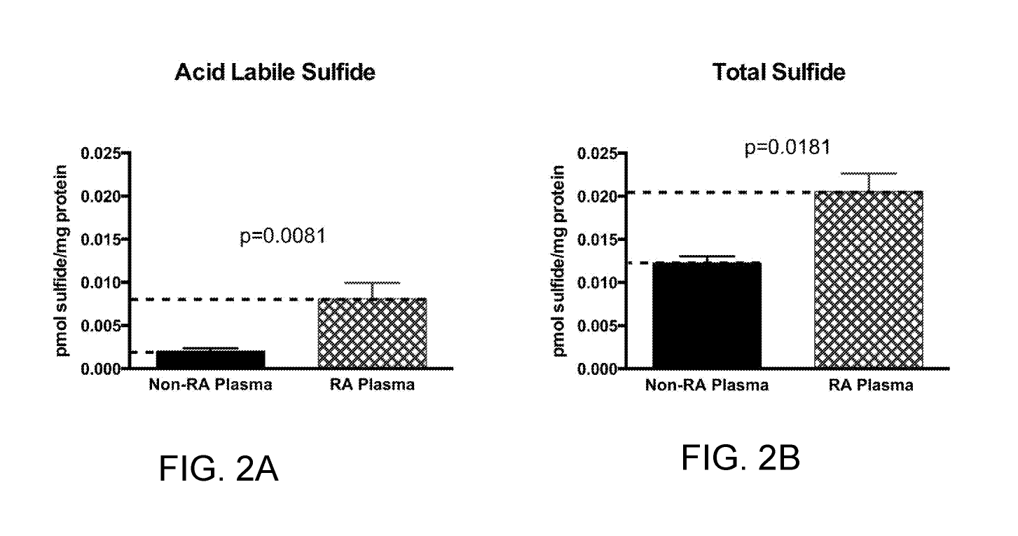

Turning to FIGS. 2A and 2B a second example is shown with further investigation of the findings in the first example. In the second example, plasma samples were collected via venipuncture from 19 patients with RA and 8 healthy subjects. Sulfide metabolites were stabilized using methods previously published by our laboratory (Peter E A, Shen X, Shah S H, Pardue S, Glawe J D, Zhang W W, Reddy P, Akkus NI, Varma J, Kevil C G. “Plasma free H2S levels are elevated in patients with cardiovascular disease,” J Am Heart Assoc. 2013 Oct. 23; 2(5):e000387. doi:10.1161/JAHA.113.000387. PubMed PMID: 24152982; PubMed Central PMCID: PMC3835249), such method fully incorporated herein. Sulfide metabolite levels were measured using MBB HPLC analytical biochemical methods as we have previously reported (Shen X, Peter E A, Bir S, Wang R, Kevil C G. “Analytical measurement of discrete hydrogen sulfide pools in biological specimens,” Free Radic Biol Med. 2012 Jun. 1-15; 52(11-12):2276-83. doi: 10.1016/j.freeradbiomed.2012.04.007. Epub 2012 Apr. 19. PubMed PMID: 22561703; PubMed Central PMCID: PMC4413934) such method fully incorporated herein. Plasma acid labile sulfide levels were significantly higher in patients with RA compared to controls. Moreover, the overall total sulfide metabolite levels were significantly elevated in plasma of RA patients compared to controls.

This disclosure serves to describe a novel method for diagnosis of RA, including noting the variance in levels between the different forms of plasma H₂S in patients with RA. By measuring levels of H₂S in the plasma instead of the synovial fluid, the option of a less invasive technique is provided to patients to monitor disease activity who may are already be suffering from joint pain. Furthermore, the discovery of the RP-HPLC technique as a way to measure the three pools of H₂S in plasma, allows a more precise, specific, and humane manner of measuring and diagnosing RA as compared to previous methods.

A further embodiment of the disclosed invention relates to the treatment of RA, as treatment of a condition is improved when the accurate diagnosis of the disease causing a symptom is achieved, and arcuate monitoring of the condition is improved..

Therapeutic agent: A substance that demonstrates some therapeutic effect by restoring or maintaining health, such as by alleviating the symptoms associated with a disease or physiological disorder, or delaying (including preventing) progression or onset of a disease. In some instances, the therapeutic agent is a chemical or pharmaceutical agent, or a prodrug. A therapeutic agent may be an agent that prevents or inhibits one or more signs or symptoms or laboratory findings associated with RA. Steroids and corticosteroids, including prednisone, prednisolone and methyprednisolone, are potent and quick-acting anti-inflammatory medications; DMARDs, an acronym for disease-modifying antirheumatic drugs, DMARDs are drugs that work to modify the course of the RA disease, include methotrexate, hydroxycholorquine, sulfasalazine, leflunomide, cyclophosphamide and azathioprine, which may be taken by mouth, self-injected or given as an infusion; Biologics, which are a subset of DMARDs, and are injected or given by infusion, and they target specific steps in the inflammatory process, they don't wipe out the entire immune response, and include abatacept (Orencia), adalimumab (Humira), anakinra (Kineret), certolizumab (Cimzia), etanercept (Enbrel), golimumab (Simponi), infliximab (Remicade), rituximab (Rituxan), tocilizumab (Actemra) and tofacitinib (Xeljanz); JAK inhibitors, a further subcategory of DMARDs that block the Janus kinase, or JAK, pathways, which are involved in the body's immune response, including Tofacitinib, and which can be taken by mouth; and NSAIDs, or nonsteroidal anti-inflammatory drugs, which can relieve pain and reduce inflammation and include ibuprofen (Advil, Motrin IB) and naproxen sodium.

A “therapeutically effective amount” or “therapeutically effective dose” is that amount or dose sufficient to inhibit or prevent onset or advancement, to treat outward symptoms, or to cause regression, of a disease. The therapeutically effective amount or dose also can be considered as that amount or dose capable of relieving symptoms caused by the disease. Thus, a therapeutically effective amount or dose of an anti-RA agent is that amount or dose sufficient to achieve a stated therapeutic effect.

While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

Wherefore, I/We claim:

1. A method for diagnosing Rheumatoid arthritis in a human patient comprising:
obtaining a plasma sample from the patient;
using an analytical instrument to determine a level of H₂S in the sample; and
diagnosing a patient with Rheumatoid arthritis when the level of H₂S in the sample is above a control level of H₂S.

2. The method of claim 1 wherein the level of H₂S in the sample and level of H₂S in the control refers to one of a level of acid labile sulfide, total sulfide, and both acid labile sulfide and total sulfide.

3. The method of claim 2 further comprising diagnosing a patient with Rheumatoid arthritis when the level of acid labile sulfide in the plasma is above one of 0.004, 0.005, 0.006, and 0.007 pmol sulfide/mg protein.

4. The method of claim 2 further comprising diagnosing a patient with Rheumatoid arthritis when the level of total sulfide in the plasma is above one of 0.014, 0.015, 0.016, 0.017, 0.018, 0.019, and 0.020 pmol sulfide/mg protein.

5. The method of claim 2 further comprising diagnosing a patient with Rheumatoid arthritis when the level of acid labile sulfide in the plasma is above one of 0.004, 0.005, 0.006, and 0.007 pmol sulfide/mg protein and the level of total sulfide in the plasma is above one of 0.014, 0.015, 0.016, 0.017, 0.018, 0.019, and 0.020 pmol sulfide/mg protein.

6. The method of claim 2 further comprising diagnosing a patient with Rheumatoid arthritis when the level of acid labile sulfide in the plasma is above one of 0.001, 0.002, 0.003, and 0.004 pmol sulfide/mg protein above the level of acid labile sulfide in the control.

7. The method of claim 2 further comprising diagnosing a patient with Rheumatoid arthritis when the level of total sulfide in the plasma is above one of 0.001, 0.002, 0.003, 0.004, 0.005, 0.006, and 0.007 pmol sulfide/mg protein above the level of total sulfide in the control.

8. The method of claim 2 further comprising diagnosing a patient with Rheumatoid arthritis when the level of acid labile sulfide in the plasma is above one of 0.001, 0.002, 0.003, and 0.004 pmol sulfide/mg protein above the level of acid labile sulfide in the control and the level of total sulfide in the plasma is above one of 0.001, 0.002, 0.003, 0.004, 0.005, 0.006, and 0.007 pmol sulfide/mg protein above the level of total sulfide in the control.

9. The method of claim 2 further comprising diagnosing a patient with Rheumatoid arthritis when the level of acid labile sulfide in the plasma one of 10%, 20% 40%, 80%, 100%, 150%, and 175% greater than the level of acid labile sulfide in the control.

10. The method of claim 2 further comprising diagnosing a patient with Rheumatoid arthritis when the level of total sulfide in the plasma one of 10%, 20% 30%, 40%, 50%, and 60% greater than the level of total sulfide in the control.

11. The method of claim 2 further comprising diagnosing a patient with Rheumatoid arthritis when the level of acid labile sulfide in the plasma one of 10%, 20% 40%, 80%, 100%, 150%, and 175% greater than the level of acid labile sulfide in the control and the level of total sulfide in the plasma one of 10%, 20% 30%, 40%, 50%, and 60% greater than the level of total sulfide in the control.

12. The method of claim 1 wherein the patient is previously suspected to have Rheumatoid arthritis based on diagnostic criteria in addition to the elevated level of H₂S, including joint pain.

13. A method for diagnosing Rheumatoid arthritis in a human patient comprising:
obtaining a plasma sample from the patient;
using an analytical instrument to determine a level of H₂S in the sample, wherein a level of H₂S refers to a level of one or more of free sulfide, acid labile sulfide, bound sulfane, and total sulfide; and
diagnosing a patient with Rheumatoid arthritis when the level of one or more of free sulfide, acid labile sulfide, bound sulfane, and total sulfide in the sample is above a respective control level of free sulfide, acid labile sulfide, bound sulfane, and total sulfide.

14. A method for diagnosing and treating Rheumatoid arthritis in a patient comprising:
analyzing a patient plasma sample for an elevated level of H2S, wherein the patient is diagnosed with RA if an elevated level of H2S is detected; and
administering pharmacologically effective dose of a therapeutic agent to the diagnosed patient.

15. The method of claim 14 wherein the therapeutic agent is one of a steroid, a corticosteroid, a disease-modifying antirheumatic drug, a biologic, a JAK inhibitor, and a nonsteroidal anti-inflammatory drug.
